(19) United States
(12) Patent Application Publication
(10) Pub. No.: US 2008/0187923 A1
(43) Pub. Date: Aug. 7, 2008

(54) Flow-Cytometric Heteroduplex Analysis for Detection of Genetic Alterations

(75) Inventors: Gabor Szabo, Debrecen (HU); Gyorgy Lustyik, Pecs (HU)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 11/910,040

(22) PCT Filed: Apr. 17, 2006
(86) PCT No.: PCT/US06/14346

§ 371 Date: Sep. 28, 2007

Related U.S. Application Data
(60) Provisional application No. 60/671,912, filed on Apr. 15, 2005.

(51) Int. Cl. C12Q 1/68

(52) U.S. Cl. 435/6

(57) ABSTRACT

The present invention provides methods and kits useful for the detection of genetic alterations and to diagnose a disease condition caused by a genetic alteration. The invention involves two different PCR products, one control “unaltered” product and one sample product, which may contain “altered” DNA, that are mixed, denatured, and allowed to re-anneal. The DNA duplexes are then treated enzymatically or chemically to cleave single stranded or mismatched DNA regions resulting from the genetic alterations. The alterations are then quantitated by measuring a decrease in a fluorescent signal resulting from removal of the fluorescent tag. Further embodiments of the invention include a second, different fluorescent tag that is used to measure non-specific degradation of the duplexes. Other embodiments of the invention include the use of avidin coated microbeads to which the duplexes may be attached for quantitation by flow cytometry. Still further embodiments include biological microbeads for this purpose.

### GOVERNMENT RIGHTS

The U.S. Government has certain rights in this invention pursuant to Grant No. R43CA96379-01 awarded by the National Cancer Institute.

### FIELD OF THE INVENTION

The invention relates to methods and kits useful for the detection of genetic alterations such as insertions, deletions and single nucleotide polymorphisms (SNPs).

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Accurate diagnostic tools specific to cancer and other illnesses are important for high quality patient care. Early detection is critical for treating many such conditions, but this can not be accomplished without a practical means for assessing an individual's propensity to develop the disease. This is particularly important for the treatment of diseases, such as various forms of cancer, in which the patient typically does not present symptoms until the disease has progressed substantially.

Identification of genetic alterations offers a tremendous advantage in analyzing the genetic basis of human disease, and the detection of changes in nucleic acid sequences such as deletions, insertions and single nucleotide polymorphisms (SNPs) have been an area of great interest in recent years. For example, mutations of certain genes have been associated with a variety of disorders-ranging from blood disorders to cancers. Genetic tests are thus becoming an increasingly important facet of medical care. Consequently, there has been an emphasis on the ability to rapidly and efficiently detect genetic alterations.

The invention described herein includes methods and kits that enable one to screen for the presence of genetic alterations, and addresses a significant need in the art.

### SUMMARY OF THE INVENTION

The following embodiments and aspects thereof are described and illustrated in conjunction with methods and kits are meant to be exemplary and illustrative, not limiting in scope.

The present invention provides methods and kits useful for the detection of genetic alterations and diagnosing disease conditions caused by genetic alterations. In one embodiment, a method for detecting a genetic alteration in a subject comprises providing an unaltered polymerase chain reaction (PCR) product with a first fluorescent marker (e.g., 6FAM, Cy3), providing a sample PCR product, using the unaltered PCR product and the sample PCR product to form a first, a second, a third and a fourth hybridization product by mixing the unaltered PCR product and the sample PCR product, denaturing (e.g., by heating, by alkaline treatment) the unaltered PCR product and the sample PCR product and annealing or hybridizing the unaltered PCR product and the sample PCR product, treating the first, the second, the third and the fourth hybridization products with a cleavage agent (e.g., a DNA modification enzyme, a chemical cleavage agent), and quantitating a fluorescent signal and/or a mean fluorescent signal from the first, the second, the third and the fourth hybridization products that have been treated with the cleavage agent to detect the genetic alteration, wherein the first and the second hybridization products are homoduplexes and the third and the fourth hybridization products are heteroduplexes.

In a further embodiment, the method comprises generating the unaltered PCR product by amplifying a wild-type or a germ line DNA and generating the sample PCR product by amplifying a DNA sample, wherein the DNA sample is homologous to the wild-type or the germ line DNA and contains or could contain a genetic alteration. The DNA sample may be obtained from a subject for whom this inventive method is employed to detect genetic alterations. Thus, the DNA sample may comprise altered DNA; or the DNA sample may not comprise any altered DNA. Generating the sample PCR product may further comprise incorporating a second fluorescent marker different from the first fluorescent marker into the altered PCR product and detecting non-specific degradation of DNA by quantitating the fluorescent signal of the second florescent marker. The second fluorescent may be, for example, 6FAM or Cy3.

In one embodiment, the DNA modification enzyme may be a nuclease, such as S1 nuclease or a cleavase. The chemical cleavage agent may be piperidine, used in conjunction with a compound capable of modifying an unpaired nucleotide in the heteroduplex; for example, hydroxylamine and potassium permanganate.

In one embodiment, an affinity tag (e.g., biotin) may be incorporated into the unaltered and the sample PCR products. In another embodiment, an affinity tag is only incorporated into the sample PCR product. The embodiments utilizing affinity tags may further comprise contacting the first, the second, the third and the fourth hybridization products to microbeads comprising avidin or streptavidin or their derivatives. Other combinations of the biotinylated primers and the two fluorescent primers may be made to all the distinction of nonspecific degradation (e.g., in homoduplexes) and specific cleavages (e.g., in heteroduplexes) and are also included in the present inventive method. Further embodiments include the use of haptens to immobilize the unaltered and/or the sample PCR products, in combination with reactants (e.g., antibodies) to the haptens.

Quantitating the fluorescent signal and/or the mean fluorescent signal may be performed using techniques such as, flow cytometry, cytofluorimetric analysis, fluorescence microscopy, confocal laser scanning microscopy and laser scanning cytometry.

In another embodiment, a method to diagnose a disease condition caused by a genetic alteration is provided wherein the method detects a genetic alteration in accordance with various embodiments of the present invention and correlates the genetic alteration with the disease condition to diagnose the disease condition.

The present invention also provides for a kit for the detection of a genetic alteration. The kit is an assemblage of materials or components. Thus, in some embodiments the kit contains compositions including a cleavage agent, a fluorescent marker, an affinity tag and/or a primer pair. In other embodiments, the kit may contain an unaltered DNA (i.e., a wt DNA) and/or an unaltered PCR product. Instructions for use may be included in the kit. “Instructions for use” typically include a tangible expression describing the technique to be employed in using the components of the kit to effect a desired outcome, such as to detect a genetic alteration. Instructions for use may include, for example: instructions to provide a DNA sample; instructions to generate a sample PCR product from the DNA sample; instructions to form a first, a second, a third and a fourth hybridization product, instructions to treating the first, the second, the third, and the fourth hybridization products with a cleavage agent; instructions to mix the unaltered PCR product and the sample PCR product, denature the unaltered PCR product and the sample PCR product (e.g., by heat, by alkaline treatment) and anneal or hybridize the unaltered PCR product and the sample PCR product; instructions to quantitate a fluorescent signal to detect a genetic alteration; instructions to use flow cytometry, cytofluorimetric analysis, fluorescence microscopy, confocal laser scanning microscopy and/or laser scanning cytometry to quantitate the fluorescent signal, instructions to incorporate a second fluorescent marker into the sample PCR product, and/or instructions to quantitate the second fluorescent signal of the second fluorescent marker to detect non-specific degradation.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in the referenced figures. It is intended that the embodiments and figures disclosed herein are to be considered illustrative rather than restrictive.

FIG. 1 depicts a multi-step process for detecting genetic alterations in accordance with an embodiment of the present invention, wherein an affinity tag is incorporated into both PCR products.

FIG. 2 depicts a multi-step process for detecting genetic alterations in accordance with an embodiment of the present invention, wherein an affinity tag is incorporated into both PCR products and a second fluorescent tag is used to measure non-specific degradation of DNA samples that may be a result of trace contaminants.

FIG. 3 depicts a multi-step process for detecting genetic alterations in accordance with an embodiment of the present invention, wherein an affinity tag is incorporated into only one of the PCR products and a second fluorescent tag is used to measure non-specific degradation of DNA samples that may be a result of trace contaminants.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, NY 1994); March, Advanced Organic Chemistry Reactions, Mechanisms and Structure 4th ed., J. Wiley & Sons (New York, NY 1992); and Sambrook and Russell, Molecular Cloning: A Laboratory Manual 3rd ed., Cold Spring Harbor Laboratory Press (Cold Spring Harbor, N.Y. 2001) provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“DNA” is meant to refer to a polymeric form of deoxyribonucleotides (i.e., adenine, guanine, thymine and cytosine) in double-stranded or single-stranded form, either relaxed or supercoiled. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the non-transcribed strand of DNA (i.e., the strand having the sequence homologous to the mRNA). The non-transcribed strand is also referred to as the “coding strand” or the “sense strand”. The complementary DNA strand, which is used as the template to produce mRNA, is referred to as the “non-coding strand” or the “antisense” strand. The term “DNA” captures molecules that include the four bases adenine, guanine, thymine and cytosine, as well as molecules that include base analogues which are known in the art.

“Disease” or “disease condition” as used herein may include, but are in no way limited to pathological conditions, whether commonly recognized as diseases or not, that relate to or that are caused by genetic alterations, including but not limited to any form of genetic polymorphism predisposing a subject to pathological conditions, including those that involve insertions or deletions of nucleotide(s) and/or rearrangements of the genome. Particular conditions and disease conditions that are believed to be appropriate to diagnose in connection with various embodiments of the present invention include conditions and disease conditions related, but are in no way limited to cancer.

A “gene” or “coding sequence” or a sequence which “encodes” a particular protein is a nucleic acid molecule that is transcribed (in the case of DNA) and translated (in the case of mRNA) into a polypeptide in vitro or in vivo when placed under the control of appropriate regulatory sequences. The boundaries of the genes are determined by a start codon at the 5′ (i.e., amino) terminus and a translation stop codon at the 3′ (i.e., carboxy) terminus. A gene can include, but is not limited to cDNA from prokaryotic or eukaryotic mRNA, genomic DNA sequences from prokaryotic or eukaryotic DNA, and even synthetic DNA sequences. A transcription termination sequence will usually be located 3′ to the gene sequence.

“Polymerase Chain Reaction” or “PCR” (U.S. Pat. No. 4,683,202) refers to a process for amplifying any desired specific nucleic acid sequence contained in a nucleic acid or mixture thereof. The process comprises treating separate complementary strands of the nucleic acid with a molar excess of two oligonucleotide primers, and extending the primers to form complementary primer extension products which act as templates for synthesizing the desired nucleic acid sequence. The steps of the reaction may be carried out stepwise or simultaneously and can be repeated as often as desired. The primers may incorporate a variety of features, including fluorescent labels, affinity tags such as biotin, avidin or streptavidin, or recognition sites for nucleases.

The term “hybridization” refers to a process by which single stranded nucleic acids are allowed to interact so that complexes or hybrids are formed by molecules with sufficiently similar, complementary sequences. Double-stranded DNA may be denatured by heat or chemical means to produce single-stranded DNA that is capable of hybridization. Hybrids can be formed by DNA, RNA, or a combination including one strand of each. Another term for the formation of DNA or RNA hybrids following denaturation is “annealing”, a process by which single stranded nucleic acids are slowly cooled allowing pairing of complementary bases to occur.

The terms “homoduplex” and “heteroduplex” refer to the products of hybridization or annealing. A homoduplex contains two nucleic acid strands wherein the strands are from the original PCR products. Homoduplexes are normally of equal length and presumably are completely complementary; i.e., no mismatches, insertions, or deletions in either strand. However, in view of the fact that polymerases, including Taq polymerase, make errors resulting mismatches, insertions, or deletions in either strand, it is thus contemplated that the term homoduplex include duplexes in which such errors exists. A heteroduplex contains two nucleic acid strands wherein one strand came from each of the original PCR products. While throughout the specification the term “heteroduplex” may be used in embodiments wherein the heteroduplex comprises an un-altered strand and an altered strand, the term “heteroduplex” as used herein is not limited to these heteroduplexes. In instances wherein the DNA sample from a subject does not contain a genetic alteration, a DNA strand provided by this DNA sample is an “unaltered” trand. When this unaltered strand is combined with a strand from a control (i.e., unaltered) DNA sample, the hybridization of these strands form a heteroduplex since each strand came from each of the original PCR products. If the sample DNA product contains altered DNA, the heteroduplexes will contain a region (e.g., a mismatch, a single stranded region) that is targeted by the cleavage agent.

“Microbeads” refer to conventional polymeric or synthetic microbeads that may be composed of a number of substances, including polystyrene, corboxyl-styrene, or other carboxylated compounds. Antibodies can be covalently attached to microbeads for immunoassay-type studies. Alternatively, PCR products may be prepared using biotinylated and fluorescent dye-labeled primers on the two ends.

“Biological microbeads” include fixed prokaryotic or eukaryotic cells (e.g., bacteria, yeast, etc.). These biological microbeads can be used in the same fashion as conventional polymeric or synthetic microbeads. See, e.g., Krupa et al., “Quantitative bead assay for hyaluronidase and heparinase I,” 319 Analytical Biochemistry 280-286 (2003); Yan et al., “Microsphere-based duplexed immunoassay for influenza virus typing by flow cytometry,” 284 J. Immunological Methods 27-38 (2004); Xu et al., “Multiplexed SNP genotyping using the Qbead system: a quantum dot-encoded microsphere-based assay,” Nucleic Acids Research, vol. 31, no. 8 (2003); and Kellar & Douglass, “Multiplexed microsphere-based flow cytometric immunoassays for human cytokines,” 279 J Immunological Methods 277-285 (2003).

The present invention provides biochemical methods for identifying multiple changes in a nucleic acid, for example, a target DNA sequence as compared to a control, or wild type (wt) sequence. These changes include insertions, deletions, and substitutions (also referred to as “single nucleotide polymorphisms” or SNP's).

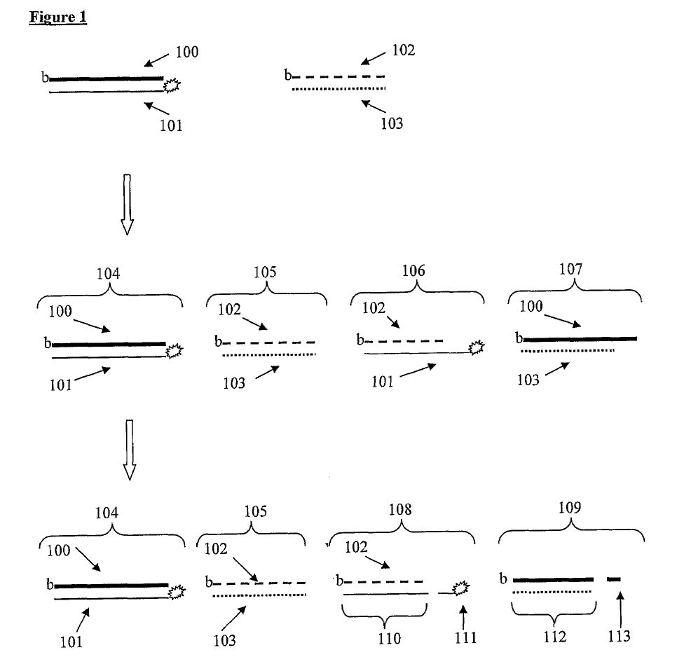

The process is generally outlined in FIGS. 1, 2 and 3. In FIG. 1, an unaltered PCR product 104 is amplified from wild-type (wt) DNA. The unaltered PCR product comprises a biotinylated wt coding strand 100 and a wt noncoding strand with a fluorescent marker 101. The altered PCR product 105 is amplified from mutant (mut) DNA, i.e., DNA that is homologous to the wt DNA but contains a genetic alteration. The altered PCR product comprises a biotinalyated mut coding strand 102 and a mut noncoding strand 103. The unaltered PCR product 104 and the altered PCR product 105 are mixed, denatured (e.g., by heat, by alkaline treatment) and annealed or hybridized to form four different hybridization products: homoduplex 1 (104), homoduplex 2 (105), heteroduplex 1 (106) and heteroduplex 2 (107). Heteroduplex 1 (106) is the only one that has the fluorescent marker and at the same time will bind to the beads and be cleaved by S1 nuclease in accordance with an embodiment of the inventive method. The duplexes 104, 105, 106 and 107 are treated with a cleaving agent, such as S1 nuclease. Treatment with S1 nuclease results in the removal of the fluorescent label at the 3′ end (see 108) resulting in a shortened heteroduplex 110, which no longer has the fluorescent label. This results in a decrease in the fluorescent signal. A heteroduplex 107 that lacks the fluorescent label due to the unaltered DNA strand being the coding strand will not result in a decrease in the fluorescent signal when treated with S1 nuclease (see 109) because the removed fragment 113 does not have a fluorescent label.

Fragments 111 and 113 may be separated from the sample. The sample may be placed in contact with avidin conjugated beads. This may be done prior to treating the sample with the cleaving agent or subsequent to treating the sample with the cleaving agent. If this is done prior to treating the sample with the cleaving agent, the biotinylated duplexes 104, 105, 106, and 107 bind to the beads. The treatment with the cleaving agent will remove fragments 111 and 113. The removal of fragment 111 results in a decrease in the fluorescent signal. If the sample is placed in contact with avidin conjugated beads subsequent to treating the sample with the cleaving agent, the biotinylated duplexes, 104, 105, 110 and 112 bind to the beads. Since fragment 111 does not bind to the beads, it will also result in a decrease in the fluorescent signal. The sample is quantitated for the fluorescent signal; for example, by flow cytometry.

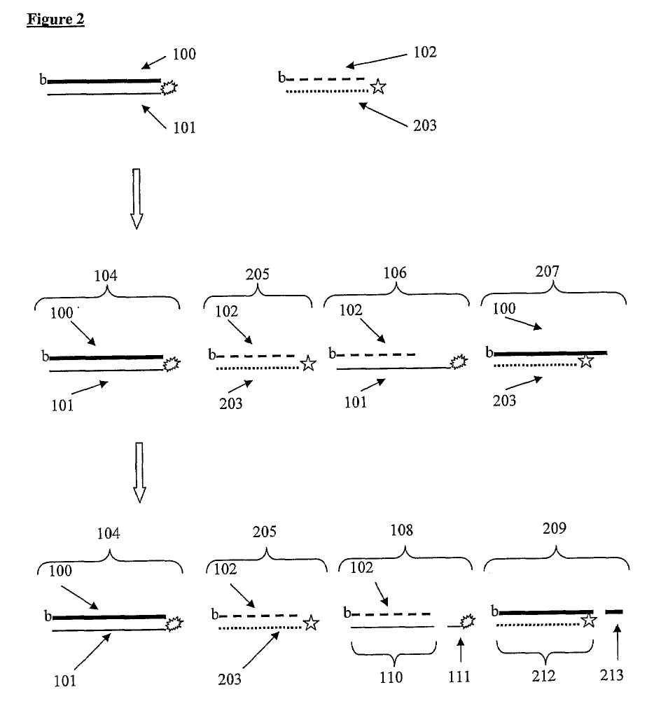

As shown in FIG. 2, a second fluorescent marker can also be used. In this embodiment, an unaltered PCR product 104 is amplified from wild-type (wt) DNA. The unaltered PCR product contains a biotinylated wt coding strand 100 and a wt noncoding strand with a fluorescent marker 101. The altered PCR product 205 is amplified from mutant (mut) DNA, i.e., DNA that is homologous to the wt DNA but contains a genetic alteration. The altered PCR product comprises a biotinalyated mut coding strand 102 and a mut noncoding strand with a second fluorescent marker 203. The unaltered PCR product 104 and the altered PCR product 205 are mixed, denatured (e.g., by heat, by alkaline treatment) and annealed or hybridized to form four different hybridization products: homoduplex 1 (104), homoduplex 2 (205), heteroduplex 1 (106) and heteroduplex 2 (207). The duplexes 104, 205, 106 and 207 are treated with a cleaving agent, such as S1 nuclease. Treatment with S1 nuclease results in the removal of the fluorescent label at the 3′ end (see 108) resulting in a shortened heteroduplex 110, which no longer has the fluorescent label. This results in a decrease in the fluorescent signal. In heteroduplexes 207 containing the coding strand of the unaltered DNA and the non-coding strand of the altered DNA, the second fluorescent tag would be present on the recessed end of the duplex and therefore will not be removed by treatment with S1 nuclease (see 209) because the removed fragment 213 does not have a fluorescent label.

In homoduplex 205, the second fluorescent marker is not removed by treatment with the cleaving agent. Thus, a decrease in this second fluorescent signal results from non-specific cleavage rather than cleavage resulting from the presence of genetic alterations.

Fragments 111 and 213 may be separated from the sample. The sample may be placed in contact with avidin conjugated beads. This may be done prior to treating the sample with the cleaving agent or after treating the sample with the cleaving agent. If this is done prior to treating the sample with the cleaving agent, the biotinylated duplexes 104, 205, 106, and 207 bind to the beads. The treatment with the cleaving agent will remove fragments 111 and 213. Removal of fragment 111 results in a decrease in the fluorescent signal. If the sample is placed in contact with avidin conjugated beads after treating the sample with the cleaving agent, the biotinylated duplexes, 104, 205, 110 and 212 bind to the beads. Since fragment 111 does not bind to the beads, it will also result in a decrease in the fluorescent signal. The sample is quantitated for the fluorescent signal; for example, by flow cytometry.

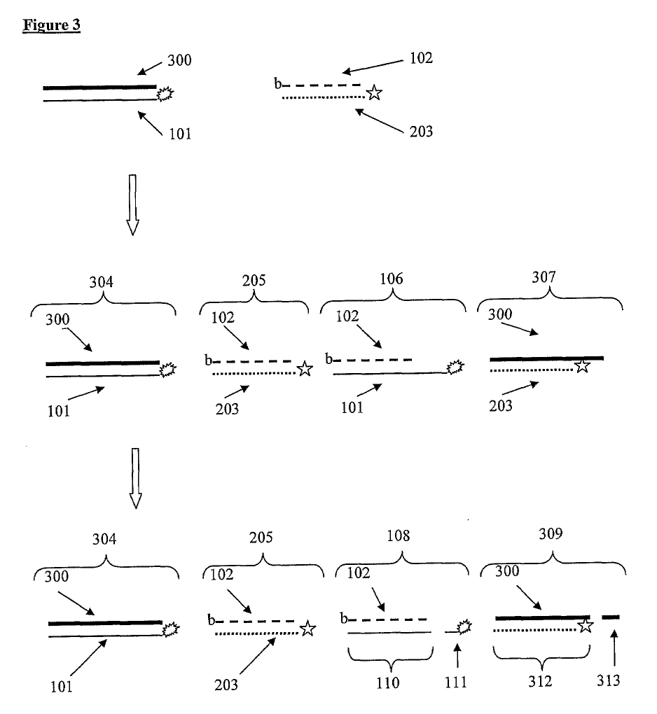

As shown in FIG. 3, only the altered PCR product comprises a biotinalyated coding strand and a second fluorescent marker is be used. In this embodiment, an unaltered PCR product 304 is amplified from wild-type (wt) DNA. The unaltered PCR product contains a wt coding strand 300 and a wt noncoding strand with a fluorescent marker 101. The altered PCR product 205 is amplified from mutant (mut) DNA, i.e., DNA that is homologous to the wt DNA but contains a genetic alteration. The altered PCR product comprises a biotinalyated mut coding strand 102 and a mut noncoding strand with a second fluorescent marker 203. The unaltered PCR product 304 and the altered PCR product 205 are mixed, denatured (e.g., by heat, by alkaline treatment, etc.) and annealed or hybridized to form four different hybridization products: homoduplex 1 (304), homoduplex 2 (205), heteroduplex 1 (106) and heteroduplex 2 (307). The duplexes 304, 205, 106 and 307 are treated with a cleaving agent, such as S1 nuclease. Treatment with S1 nuclease results in the removal of the fluorescent label at the 3′ end (see 108) resulting in a shortened heteroduplex 110, which no longer has the fluorescent label. This results in a decrease in the fluorescent signal. In heteroduplexes 307 containing the coding strand of the unaltered DNA and the non-coding strand of the altered DNA, the second fluorescent tag would be present on the recessed end of the duplex and therefore will not be removed by treatment with S1 nuclease (see 309) because the removed fragment 313 does not have a fluorescent label. However, this latter cleavage is not detected when using microbeads because fragment 307, without the biotin, does not bind to microbeads conjugated with avidin or a derivative of avidin.

In homoduplex 205, the second fluorescent marker is not removed by treatment with the cleaving agent. Thus, a decrease in this second fluorescent signal results from non-specific cleavage rather than cleavage resulting from the presence of genetic alterations.

Fragments 304, 307, 111, 312, and 313 may be separated from the sample. The sample may be placed in contact with avidin conjugated beads. This may be done prior to treating the sample with the cleaving agent or after treating the sample with the cleaving agent. If this is done prior to treating the sample with the cleaving agent, the biotinylated duplexes 205 and 106 bind to the beads. The treatment with the cleaving agent will remove fragment 111. Removal of fragment 111 results in a decrease in the fluorescent signal. If the sample is placed in contact with avidin conjugated beads after treating the sample with the cleaving agent, the biotinylated duplexes, 205 and 110 bind to the beads. Since fragment 111 does not bind to the beads, it will also result in a decrease in the fluorescent signal. The sample is quantitated for the fluorescent signal; for example, by flow cytometry.

A DNA sample that is to be tested for the presence of a genetic alteration may contain the mut DNA which is amplified to generate a sample DNA product which may contain the “altered” product. The DNA sample that is to be tested for the presence of a genetic alteration may contain normal DNA which is amplified to generate a sample DNA product which may contain and “unaltered” product. Each product can be further defined to comprise a coding strand and a non-coding strand. This is an arbitrary designation for the purposes of clarity in this application; genetic alterations in either the coding or non-coding strand can be measured using this method.

A control unaltered PCR product may contain a fluorescent marker such as 6 FAM or Cy3 on the 3′ end of the non-coding DNA strand. In an alternative embodiment, the coding DNA strand may contain a fluorescent marker. The two PCR products are mixed, denatured (e.g., by heat, by alkaline treatment), and annealed or hybridized to form hybrids. Upon annealing or hybridization, four different hybridization products are formed; the original sample PCR product and the original control unaltered PCR product (homoduplexes), and two heteroduplexes in which one strand came from each of the original PCR products. Since the sample DNA may or may not have a genetic alteration, i.e., be “altered”, there are four possible heteroduplexes that can be formed. In instances where the sample DNA product contains altered DNA, one heteroduplex is an un-altered control coding strand paired with an altered sample DNA non-coding strand, and one is an altered sample DNA coding strand paired with an un-altered control non-coding strand. In instances where the sample DNA product contains un-altered DNA, one heteroduplex is an un-altered control coding strand paired with an un-altered sample DNA non-coding strand and one is an un-altered sample DNA coding strand paired with an un-altered control non-coding strand. While throughout the specification the term “heteroduplex” may be used in embodiments wherein the heteroduplex comprises an un-altered strand and an altered strand, the term “heteroduplex” as used herein is not limited to such heteroduplexes. In instances wherein the DNA sample from a subject does not contain a genetic alteration, a DNA strand provided by this DNA sample is an “unaltered” strand. When this unaltered strand is combined with a strand from a control (i.e., unaltered) DNA sample, the hybridization of these strands form a heteroduplex since each strand came from each of the original PCR products. If the sample DNA product contains altered DNA, the heteroduplexes will contain a region (e.g., a mismatch, a single stranded region) that is targeted by the cleavage agent.

The homo- and heteroduplexes are then treated with a cleaving agent (e.g., a DNA modification enzyme such as S1 nuclease). S1 nuclease degrades single stranded DNA from the ends of DNA strands or at regions of paired DNA where a mismatch creates a single-stranded “bubble” or alternative DNA structures such as hairpins, etc. In situations where the altered DNA strand is shorter, or contains mismatches of two or more bases, treatment with S1 nuclease results in removal of the fluorescent label at the 3′ end of the unaltered, non-coding strand of DNA. In an embodiment wherein the fluorescent label is on the unaltered, coding strand of DNA, treatment with S1 nuclease results in removal of the fluorescent label at the 3′ end of the coding strand. The removal of the fluorescent label by the nuclease results in a decrease in the fluorescent signal associated with the heteroduplex. The fragments cleaved by the cleavage agent may be separated from the sample prior to quantitating the fluorescent signal. This may be accomplished by the use of an affinity tag as discussed below. The goal of the process is to quantitate the decrease in this fluorescent signal as a result of the presence of altered DNA as part of heteroduplexes. In one embodiment, the fluorescent signal is quantitiated from the immobilized hybridization products. In another embodiment, the fluorescent signal may be quantitated from the part of the sample that is not immobilized. In addition to nucleases, other DNA modification enzymes, such as cleavases, may be suitable.

The homoduplexes will not exhibit a decrease in fluorescent signal, as they presumably are completely complementary to each other (aside from possible random errors made by the polymerase used) and therefore are not substrates for S1 nuclease. Additionally, heteroduplexes that lack the fluorescent tag because the un-altered DNA strand is the coding strand will not result in a decrease in the fluorescent signal.

The degree of genetic alteration may be assessed by comparing the fluorescent signal in the treated sample with the signal produced by an untreated control sample. In another embodiment, the detection of genetic alterations may be assessed by a comparison between the fluorescent signal of the sample prior to treatment with the cleaving agent and the fluorescent signal of the sample subsequent to treatment with the cleaving agent. Quantitating the fluorescent signal may be performed, for example, by flow cytometry, cytofluorimetric analysis, fluorescence microscopy, confocal laser scanning microscopy, and/or laser scanning cytometry, allowing for the determination of the fluorescent signal or the average (e.g., mean, median, mode, etc.) fluorescent signal.

An additional feature of the invention is the use of a second, different fluorescent tag that measures the non-specific degradation of DNA samples that may result from trace contaminants. This second fluorescent tag, which is necessarily different from the fluorescent tag described earlier (for example, one is 6 FAM and the other is Cy3), may be incorporated into a sample (e.g., altered) DNA strand. In homoduplexes containing the coding strand of the altered DNA, and the non-coding strand of the altered DNA, the second fluorescent tag would not be removed by treatment with S1 nuclease. Therefore, a decrease in the second fluorescent signal results from non-specific cleavage rather than cleavage resulting from the presence of genetic alterations. The use of the two fluorescent labels simultaneously permits the measurement of specific, mismatch-related cleavages and non-specific cleavage as a result of background nucleic acid degradation.

Another embodiment of the invention includes an affinity tag such as biotin incorporated into one or more of the primers used to generate the original control and sample PCR products. The biotin tag allows the hybridization products to be attached to support matrices (e.g., microbeads) that are conjugated with avidin or any of its derivatives or avidin-like proteins (e.g., extravidin, streptavidin, etc.) and subsequent quantitation of the fluorescent labels by flow cytometry. Biological microbeads may also be used for this purpose. Other affinity tags may be used in alternate embodiments of the invention. Quantization of the released and fluorescent dye-labeled fragments (e.g., by spectrofluorimetry) will also allow simultaneous determination of specific versus nonspecific degradation, thereby determining the presence or absence of genetic alterations.

Additional embodiments include the use of haptens to immobilize the unaltered and/or the sample PCR products, in combination with reactants (e.g., antibodies) to the haptens.

Further embodiments of the invention include the use of chemical cleavage agents, such as piperidine in combination with a compound that modifies unpaired nucleotides, such as hydroxylamine and potassium permanganate, to detect single nucleotide mismatches. Hydroxylamine modifies unpaired cytosines, and potassium pennanganate modifies unpaired thymines. Piperidine then cleaves the modified DNA strands. Other chemical cleavage agents and compounds may be used in connection with alternate embodiments of the present invention. These treatments result in the removal of the fluorescent tag and a quantifiable decrease in the fluorescent signal, and therefore provide a way to observe the presence of single base pair substitutions. In cases of SNPs and mutations that involve the exchange of nucleotide to another, the sensitivity of the inventive method may depend on the particular case, as ajoint result of the sensitivity of either the enzymatic or the chemical cleavage reactions to the SNPs and/or mutations and the error rate of the polymerase used. Other embodiments of the invention are kits designed to detect genetic alterations associated with a particular disease or physiologic condition that does not constitute a disease utilizing the reagents and methods described above. Such kits may provide, for example, a powerful diagnostic tool for use in genetic testing and other applications. The kits are an assemblage of materials or components. Thus, in some embodiments the kit contains compositions including a cleavage agent, a fluorescent marker, an affinity tag, and/or a primer pair as described above. In other embodiments, the kit may contain a control sample comprising unaltered DNA and/or an unaltered PCR product. The exact nature of the components configured in the inventive kit depends on its intended purpose. For example, some embodiments are configured for the purpose of detecting a genetic alteration. Other embodiments are configured for the purpose of detecting single nucleotide mismatches. In one embodiment, the kit is configured particularly for the purpose of detecting genetic alterations in mammalian subjects. In another embodiment, the kit is configured particularly for the purpose of detection in human subjects. In further embodiments, the kit is configured for veterinary applications, detection in subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to detect a genetic alteration. Instructions for use may include, for example: instructions to provide a DNA sample; instructions to generate a sample PCR product from the DNA sample; instructions to form a first, a second, a third and a fourth hybridization product; instructions to treating the first, the second, the third, and the fourth hybridization products with a cleavage agent; instructions to mix the unaltered PCR product (i.e., the control) and the sample PCR product, denature the unaltered PCR product and the sample PCR product and anneal or hybridize the unaltered PCR product and the sample PCR product; instructions to quantitate a fluorescent signal to detect a genetic alteration; instructions to use flow cytometry, cytofluorimetric analysis, fluorescence microscopy, confocal laser scanning microscopy, and/or laser scanning cytometry; instructions to incorporate a second fluorescent marker into the sample PCR product; and/or instructions to quantitate the second fluorescent marker to detect non-specific degradation. Optionally, the kit also contains other useful components, such as test tubes, support matrices, microbeads, biological microbeads, diluents, buffers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit. The packaging material is constructed by well known methods, preferably to provide a sterile, contaminant-free environment. The packaging materials employed in the kit are those customarily utilized in genetic testing. As used herein, the term “package” refers to a suitable solid matrix or material such as glass, plastic, paper, foil, and the like, capable of holding the individual kit components.

The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention.

One skilled in the art may develop equivalent means or reactants without the exercise of inventive capacity and without departing from the scope of the invention.

### Example 1

### Detecting Genetic Alterations

Referring to FIG. 1, an unaltered PCR product 104 is amplified from wild-type (wt) DNA. The unaltered PCR product comprises a biotinylated wt coding strand 100 and a wt noncoding strand with a fluorescent marker 101. The altered PCR product 105 is amplified from mutant (mut) DNA, i.e., DNA that is homologous to the wt DNA but contains a genetic alteration. The altered PCR product comprises a biotinalyated mut coding strand 102 and a mut noncoding strand 103. The unaltered PCR product 104 and the altered PCR product 105 are mixed, denatured (e.g., by heat, by alkaline treatment) and annealed or hybridized to form four different hybridization products: homoduplex 1 (104), homoduplex 2 (105), heteroduplex 1 (106) and heteroduplex 2 (107). Heteroduplex 1 (106) is the only one that has the fluorescent marker and at the same time will bind to the beads and be cleaved by S1 nuclease in accordance with an embodiment of the inventive method. The duplexes 104, 105, 106 and 107 are treated with a cleaving agent, such as S1 nuclease. Treatment with S1 nuclease results in the removal of the fluorescent label at the 3′ end (see 108) resulting in a shortened heteroduplex 110, which no longer has the fluorescent label. This results in a decrease in the fluorescent signal. A heteroduplex 107 that lacks the fluorescent label due to the unaltered DNA strand being the coding strand will not result in a decrease in the fluorescent signal when treated with S1 nuclease (see 109) because the removed fragment 113 does not have a fluorescent label.

Fragments 111 and 113 may be separated from the sample. The sample may be placed in contact with avidin conjugated beads. This may be done prior to treating the sample with the cleaving agent or after treating the sample with the cleaving agent. If this is done prior to treating the sample with the cleaving agent, the biotinylated duplexes 104, 105, 106, and 107 bind to the beads. The treatment with the cleaving agent will remove fragments 111 and 113. Removal of fragment 111 results in a decrease in the fluorescent signal. If the sample is placed in contact with avidin conjugated beads after treating the sample with the cleaving agent, the biotinylated duplexes, 104, 105, 110 and 112 bind to the beads. Since fragment 111 does not bind to the beads, it will also result in a decrease in the fluorescent signal. The sample is quantitated for the fluorescent signal; for example, by flow cytometry.

### Example 2

### Detecting Genetic Alterations Wherein a Second Fluorescent Tag is Used to Measure Non-Specific Degradation of DNA Samples that May be a Result of the Breathing of DNA or Trace Nuclease Contaminants

Referring to FIG. 2, an unaltered PCR product 104 is amplified from wild-type (wt) DNA. The unaltered PCR product contains a biotinylated wt coding strand 100 and a wt noncoding strand with a fluorescent marker 101. The altered PCR product 205 is amplified from mutant (mut) DNA, i.e., DNA that is homologous to the wt DNA but contains a genetic alteration. The altered PCR product comprises a biotinalyated mut coding strand 102 and a mut noncoding strand with a second fluorescent marker 203. The unaltered PCR product 104 and the altered PCR product 205 are mixed, denatured (e.g., by heat, by alkaline treatment) and annealed or hybridized to form four different hybridization products: homoduplex 1 (104), homoduplex 2 (205), heteroduplex 1 (106) and heteroduplex 2 (207). The duplexes 104, 205, 106 and 207 are treated with a cleaving agent, such as S1 nuclease. Treatment with S1 nuclease results in the removal of the fluorescent label at the 3′ end (see 108) resulting in a shortened heteroduplex 110, which no longer has the fluorescent label. This results in a decrease in the fluorescent signal. In heteroduplexes 207 containing the coding strand of the unaltered DNA and the non-coding strand of the altered DNA, the second fluorescent tag would be present on the recessed end of the duplex and therefore will not be removed by treatment with Si nuclease (see 209) because the removed fragment 213 does not have a fluorescent label.

In homoduplex 205, the second fluorescent marker is not removed by treatment with the cleaving agent. Thus, a decrease in this second fluorescent signal results from non-specific cleavage rather than cleavage resulting from the presence of genetic alterations.

Fragments 111 and 213 may be separated from the sample. The sample may be placed in contact with avidin conjugated beads. This may be done prior to treating the sample with the cleaving agent or after treating the sample with the cleaving agent. If this is done prior to treating the sample with the cleaving agent, the biotinylated duplexes 104, 205, 106, and 207 bind to the beads. The treatment with the cleaving agent will remove fragments 111 and 213. Removal of fragment 111 results in a decrease in the fluorescent signal. If the sample is placed in contact with avidin conjugated beads after treating the sample with the cleaving agent, the biotinylated duplexes, 104, 205, 110 and 212 bind to the beads. Since fragment 111 does not bind to the beads, it will also result in a decrease in the fluorescent signal. The sample is quantitated for the fluorescent signal; for example, by flow cytometry.

### EXAMPLE 3

### Detecting Genetic Alterations Wherein Only One PCR Product is Biotinylated and a Second Fluorescent Tag is Used to Measure Non-Specific Degradation of DNA Samples that May be a Result of the Breathing of DNA or Trace Nuclease Contaminants

Referring to FIG. 3, only the altered PCR product comprises a biotinalyated coding strand and a second fluorescent marker is be used. In this embodiment, an unaltered PCR product 304 is amplified from wild-type (wt) DNA. The unaltered PCR product contains a wt coding strand 300 and a wt noncoding strand with a fluorescent marker 101. The altered PCR product 205 is amplified from mutant (mut) DNA, i.e., DNA that is homologous to the wt DNA but contains a genetic alteration. The altered PCR product comprises a biotinalyated mut coding strand 102 and a mut noncoding strand with a second fluorescent marker 203. The unaltered PCR product 304 and the altered PCR product 205 are mixed, denatured (e.g., by heat, by alkaline treatment, etc.) and annealed or hybridized to form four different hybridization products: homoduplex 1 (304), homoduplex 2 (205), heteroduplex 1 (106) and heteroduplex 2 (307). The duplexes 304, 205, 106 and 307 are treated with a cleaving agent, such as S1
1 nuclease results in the removal of the fluorescent label at the 3′ end (see 108) resulting in a shortened heteroduplex 110, which no longer has the fluorescent label. This results in a decrease in the fluorescent signal. In heteroduplexes 307 containing the coding strand of the unaltered DNA and the non-coding strand of the altered DNA, the second fluorescent tag would be present on the recessed end of the duplex and therefore will not be removed by treatment with S1 nuclease (see 309) because the removed fragment 313 does not have a fluorescent label. However, this latter cleavage is not detected when using microbeads because fragment 307, without the biotin, does not bind to microbeads conjugated with avidin or a derivative of avidin.

In homoduplex 205, the second fluorescent marker is not removed by treatment with the cleaving agent. Thus, a decrease in this second fluorescent signal results from non-specific cleavage rather than cleavage resulting from the presence of genetic alterations.

Fragments 304, 307, 111, 312, and 313 may be separated from the sample. The sample may be placed in contact with avidin conjugated beads. This may be done prior to treating the sample with the cleaving agent or after treating the sample with the cleaving agent. If this is done prior to treating the sample with the cleaving agent, the biotinylated duplexes 205 and 106 bind to the beads. The treatment with the cleaving agent will remove fragment 111. Removal of fragment 111 results in a decrease in the fluorescent signal. If the sample is placed in contact with avidin conjugated beads after treating the sample with the cleaving agent, the biotinylated duplexes, 205 and 110 bind to the beads. Since fragment 111 does not bind to the beads, it will also result in a decrease in the fluorescent signal. The sample is quantitated for the fluorescent signal; for example, by flow cytometry.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A method for detecting a genetic alteration in a subject, comprising:
providing an unaltered polymerase chain reaction (PCR) product with a first fluorescent marker:
providing a sample PCR product;
using the unaltered PCR product and the sample PCR product to form a first, a second, a third and a fourth hybridization product by mixing the unaltered PCR product and the sample PCR product, denaturing the unaltered PCR product and the sample PCR product;
treating the first, the second, the third and the fourth hybridization products with a cleavage agent selected from the group consisting of a DNA modification enzyme, a chemical cleavage agent and combinations thereof; and
quantitating a first fluorescent signal from the first, the second, the third and the fourth hybridization products that have been treated with the cleavage agent to detect the genetic alteration,
wherein the first and the second hybridization products are homoduplexes and the third and the fourth hybridization products are heteroduplexes.

2. The method of claim 1, further comprising:
generating the unaltered PCR product by amplifying a wild-type or a germ line DNA; and
generating the sample PCR product by amplifying a DNA sample from the subject, wherein the DNA sample is homologous to the wild-type or the germ line DNA and contains or could contain a genetic alteration.

3. The method of claim 1, wherein the DNA modification enzyme is selected from the group consisting of a nuclease, a cleavase and combinations thereof.

4. The method of claim 1, wherein the first florescent marker is selected from the group consisting of 6 FAM, Cy3 and combinations thereof.

5. The method of claim 2, wherein generating the sample PCR product further comprises:
incorporating a second fluorescent marker different from the first fluorescent marker into the sample PCR product; and
detecting the non-specific degradation of DNA by quantiating the fluorescent signal fluorescent marker.

6. The method of claim 5, wherein the second fluorescent marker is selected from the group consisting of 6 FAM, Cy3 and combinations thereof.

7. The method of claim 2, wherein generating the unaltered PCR product and/or the sample PCR product comprises:
incorporating an affinity tag into the unaltered PCR product and/or the sample PCR product.

8. The method of claim 7, wherein the affinity tag is biotin.

9. The method of claim 8, further comprising:
contacting the first, the second, the third and the fourth hybridization products to a support matrix comprising avidin or a derivative thereof.

10. The method of claim 9, wherein the support matrix comprises microbeads.

11. The method of claim 1, wherein quantitating the fluorescent signal is performed using a technique selected from the group consisting of flow cytometry, cytofluorimetric analysis, fluorescence microscopy, confocal laser scanning microscopy, laser scanning cytometry and combinations thereof.

12. The method of claim 1, wherein the chemical cleavage agent is piperidine.

13. The method of claim 12, further comprising:
using a compound capable of modifying an unpaired nucleotide,
wherein if the third and/or the fourth hybridization products have an unpaired nucleotide, the unpaired nucleotide is modified by the compound.

14. The method of claim 13, wherein the compound capable of modifying an unpaired nucleotide is selected from the group consisting of hydroxylamine, potassium permanganate and combinations thereof.

15. The method of claim 1, further comprising:
using a hapten in combination with reactants to immobilize the unaltered and/or the sample PCR products to a support matrix.

16. A kit for the detection of a genetic alteration in a subject, comprising:
a cleavage agent selected from the group consisting of a DNA modification enzyme, a chemical cleavage agent and combinations thereof;
a first fluorescent marker;
an affinity tag;
a primer pair; and
instruction to use the cleavage agent, the first fluorescent marker, the affinity tag detect the genetic alteration.

17. The kit of claim 16, further comprising:
an unaltered polymerase chain reaction (PCR) product selected from the group consisting of an unaltered PCR product with a first fluorescent marker, an unaltered PCR product with a first fluorescent marker and an affinity tag and combinations thereof.

18. The kit of claim 16, wherein the instructions comprise:
instructions to provide a DNA sample from the subject;
instructions to use the primer pair and the affinity tag to generate a sample PCR product by amplifying the DNA sample;
instructions to use the unaltered PCR product and the sample PCR product to form a first, a second, a third and a fourth hybridization product by mixing the unaltered PCR product and the sample PCR product, denaturing the unaltered PCR product and the sample PCR product, and annealing or hybridizing the unaltered PCR product and the sample PCR product;
instructions to treat the first, the second, the third and the fourth hybridization product with the cleavage agent; and
instructions to quantitate a fluorescent signal of the first fluorescent marker from the first, the second, the third and the fourth hybridization products that have been treated with the cleavage agent to detect the genetic alteration,
wherein the first and the second hybridization product are homoduplexes and the third and the fourth hybridization products are heteroduplexes.

19. The kit of claim 17, wherein the unaltered PCR product is amplified from a wild-type or a germ line DNA.

20. The kit of claim 16, wherein the DNA modification enzyme is selected from the group consisting of a nuclease, a cleavase and combinations thereof.

21. The kit of claim 16, wherein the first fluorescent marker is selected from the group consisting of 6 FAM, Cy3 and combinations thereof.

22. The kit of claim 16, further comprising:
a second fluorescent marker;
instructions to incorporate the second fluorescent marker into a sample PCR product; and
instructions to detect non-specific degradation of DNA by quantitating the fluorescent signal fo the second fluorescent marker,
wherein the first and the second fluorescent marker are different.

23. The kit of claim 22, wherein the second fluorescent marker is selected from the group consisting of 6 FAM, Cy3 and combination thereof.

24. The kit of claim 16, wherein the affinity tag is biotin.

25. The kit of claim 24, further comprising instructions to contact the first, the second, the third and the fourth hybridization products to a support matrix comprising avidin or a derivative thereof.

26. The kit of claim 25, wherein the support matrix comprises microbeads.

27. The kit of claim 18, wherein instructions to quantitate the fluorescent signal of the first fluorescent marker comprise instructions to use a technique selected from the group consisting of flow cytometry, cytofluorimetric analysis, fluorescence microscopy, confocal laser scanning microscopy, laser scanning cytometry and combinations thereof.

28. The kit of claim 16, wherein the chemical cleavage agent is piperidine.

29. The kit of claim 28, further comprising:
a compound capable of modifying an unpaired nucleotide; and
instructions to use the compound,
wherein if the third and/or the fourth hybridization products have an unpaired nucleotide, the compound modifies the unpaired nucleotide.

30. The kit of claim 29, wherein the compound capable of modifying the unpaired nucleotide is selected from the group consisting of hydroxylamine, potassium permanganate and combinations thereof.

31. The kit of claim 16, further comprising:
a quantity of a hapten; and instructions to use the hapten in combination to immobilize the unaltered and/or the sample PCR products to a solid support matrix.

32. A method to diagnose a disease condition caused by a genetic alteration, comprising:
providing an unaltered polymerase chain reaction (PCR) product with a first fluorescent marker;
using the unaltered PCR product and the sample PCR product to form a first, a second, a third and a fourth hybridization product by mixing the unaltered PCR product and the sample PCR product; denaturing the unaltered PCR product and the sample PCR product, and annealing or hybridizing the unaltered: PCR product and the sample PCR product;
